(19) United States
(12) Patent Application Publication
(10) Pub. No.: US 2025/0035592 A1
(43) Pub. Date: Jan. 30, 2025

(54) METHOD AND DEVICE FOR PROCESSING AN ELECTRICAL SIGNAL FOR AN ULTRASONIC TRANSMITTING DEVICE

(71) Applicant: SUPERSONIC IMAGINE, Aix-En-Provence (FR)

(72) Inventors: Laurent MARSAC, Aix-en-Provence (FR); Cyril DOLGOFF, Ginasservis (FR); Bo ZHANG, Simiane-Collongue (FR); Thomas FRAPPART, Aix-en-Provence (FR)

(21) Appl. No.: 18/754,924

(22) Filed: Jun. 26, 2024

(30) Foreign Application Priority Data
Jul. 27, 2023 (FR) 2308112

(51) Int. Cl. G01N 29/34; G01N 29/06

(52) U.S. Cl. CPC G01N 29/346; G01N 29/069; G01N 29/343

(57) ABSTRACT

The present invention relates to a device (10) and a method for processing an electrical signal (SG1) for an ultrasonic transmitting device (20). The method comprises: generating an electrical signal (SG1) defining ultrasonic waves (W), the periodicity of the electrical signal (SG1) being modified by modulation (ML1) of the phase of the signal (PH1); and supplying the electrical signal (SG1) to the ultrasonic transmitting device (20) to cause the ultrasonic waves (W) to be emitted.

### PRIOR ART

An ultrasonic transmitting device (or ultrasonic transmission device) usually comprises a plurality of transducer elements configured to emit ultrasonic waves for a variety of purposes, for example for ultrasound imaging.

To achieve this, the ultrasonic transmitting device can be controlled by means of electrical signals, which can be emitted by means of one or more pulsers or linear amplifiers. These electrical signals define waves transmitted to the transducer elements of the transmitting device, which causes ultrasonic waves to be emitted into a given medium. Electrical signals may be produced in response by these same transducer elements (or other transducer elements), these signals representing a response (or echo) from the medium to the wave stimuli.

In general, during operation, such an ultrasonic transmitting device emits electromagnetic waves (EM). In this respect, the ultrasonic transmitting device also constitutes an electromagnetic wave emitting device (also known as an EM emitting device). However, high levels of electromagnetic radiation are not always desirable.

There are thus various standards that impose authorised limit levels for electromagnetic emissions from electrical, scientific and medical equipment, such as IEC 60601-1-2, NF EN 55011/CISPR 11 and IEC 61000-4-3.

In particular, the CISPR 11 standard (cf. for example edition 6.2 2019 January) defines the authorised limits (i.e. the “quasi-peak’” values of electromagnetic emission) for the different frequency ranges used. Consequently, the energy of an electric field emitted by a device must not exceed these limits in order to be declared a device that complies with said standard. For example, table 6 of the CISPR 11 standard covers electromagnetic radiation disturbance limits for Class A Group 1 equipment, measured on a test site. Moreover, table 2 of the CISPR 11 standard covers disturbance voltage limits for Class A Group 1 equipment, measured on a test site. A medical device, such as a system comprising an ultrasonic probe, is typically Class A in the sense that it is a non-domestic appliance.

In addition, the IEC 61000-4-3/EN 61000-4-3 standard defines an electromagnetic compatibility (EMC) standard. Parts 4-3 in particular cover the test and measurement techniques used, as well as the immunity test to radiated electromagnetic fields at radio frequencies.

Since ultrasonic devices emit electromagnetic waves by design, they must remain compatible with said standards, whilst at the same time enabling optimum use, i.e. emission of waves useful for the study of the region of interest concerned.

### DESCRIPTION OF THE INVENTION

As mentioned above, conventional ultrasonic transmitting devices are in particular problematic in that they can be the source of electromagnetic emissions that might need to be limited, for example in their radiation, directions and/or intensity.

One of the objects of the present invention is to address at least one of the issues or shortcomings described above.

In particular, one object of the present invention is to limit the electromagnetic emissions of an ultrasonic transmitting device whilst at the same time ensuring that it performs adequately, for example in terms of spectral response and/or acoustic energy (or power) supplied.

In particular, one object of the present invention is to develop an efficient ultrasonic transmitting device that generates minimum electromagnetic emissions during operation and has limited complexity in terms of design, manufacturing and use.

To this end, according to a first aspect, the present invention relates to a method for processing an electrical signal for an ultrasonic transmitting device, said method comprising:
generating an electrical signal defining ultrasonic waves, the periodicity of the electrical signal being modified by modulation of the phase of the signal; and
supplying the electrical signal to the ultrasonic transmitting device to cause the ultrasonic waves to be emitted.

Implementing such a method advantageously makes it possible to limit the electromagnetic radiation emitted by an ultrasonic transmitting device whilst at the same time ensuring that it performs adequately, for example in terms of spectral response and/or acoustic energy (or power) supplied. It is thus advantageously possible to limit the level of electromagnetic emissions with minimal impact (ideally no impact) on the desired operating performance of the ultrasonic transmitting device.

To do this, the electrical signal supplied to the ultrasonic transmitting device is adapted or modified by modulation of the phase of the signal. By disturbing the phase of the electrical signal, it is advantageously possible to reduce the harmonics contained in this signal and thus minimise electromagnetic (EM) radiation. This modulation can advantageously be carried out in an existing transmitting system or device without the need for additional devices and/or structural modifications to the existing system or device. The complexity and costs involved in carrying out the method can therefore be limited.

Electromagnetic emissions can be limited in the various operating modes of the ultrasonic transmitting device. Examples of different operating modes of a transmitting device in the form of an ultrasonic probe can comprise a B-mode (i.e. brightness mode), a Doppler mode or a Shear-Wave mode (i.e. shear wave elastography mode). In other words, it is not necessary to change or modify the specific operating mode of an ultrasonic transmitting device to obtain reductions in electromagnetic emissions according to the notion of the present invention.

Such reductions in electromagnetic emissions can thus be attained independently of the selected operating mode, i.e. whatever the operating mode of the device.

The method according to the invention can include other features that can be taken separately or in combination, in particular among the embodiments that follow which are set out purely by way of example and can be combined or associated unless stated otherwise.

According to one example, the modulation of the phase of the signal is configured to cause the frequency spectrum of the electrical signal to broaden.

According to one example, modulation of the phase of the electrical signal is deterministic over time.

According to one example, modulation of the phase of the electrical signal is random over time.

According to one example, modulation of the phase of the electrical signal comprises:
introducing, into the electrical signal, at least one delay corresponding to a period during which the voltage of the electrical signal is kept at a constant value.

According to one example, said at least one delay is strictly less than a half period of the electrical signal.

According to one example, at least 4 delays are introduced into the electrical signal.

According to one example, the electrical signal comprises a plurality of successive sequences each comprising at least one wave cycle of the electrical signal, a delay separating each pair of consecutive sequences, the electrical signal generated during said sequences being in phase.

According to one example, modulation of the phase of the electrical signal comprises:
an angular phase shift of the electrical signal in at least one cycle of the electrical signal.

According to one example, the angular shift is configured to cause a phase inversion of the electrical signal.

According to one example, an angular shift is introduced at each half period of the electrical signal.

According to one example, the angular shift introduced into the electrical signal increases, or decreases, gradually over time. According to one example, the electrical signal comprises at least one first period (or first phase) during which the angular shift introduced into the electrical signal increases gradually, and at least one second period (or second phase) during which the angular shift introduced into the electrical signal decreases gradually.

According to one particular example, the electrical signal modified by said modulation comprises at least one of:
at least one first phase during which the angular shift introduced into the electrical signal increases gradually; and
at least one second phase during which the angular shift introduced into the electrical signal decreases gradually.

According to one example, modulation of the phase of the electrical signal comprises a variation in the period of the cycles of the electrical signal over time.

According to one example, modulation of the phase is configured so that the electrical signal supplies electrical energy at least equal to theoretical energy that would be supplied by the electrical signal if its phase were not disturbed by said phase modulation.

According to one example, the phase is modulated while maintaining the same frequency on each cycle of the electrical signal.

According to one example, the ultrasonic waves are compression waves generating shear waves in a medium.

According to one example, said method is applied to medical ultrasound imaging.

According to another aspect, the present invention can involve a computer program comprising instructions which carry out the method according to the first aspect when the program is run by a computer. In particular, the various steps of the method according to the first aspect can be defined by computer program instructions.

Such a computer program can use any programming language or equivalent, and it can be in the form of a source code, object code, or intermediate code between a source code and object code, such as in a partially compiled form, or in any other desirable form.

According to another aspect, the present invention relates to a recording medium (or storage medium), readable by a computer (or a processor), on which a computer program according to this same aspect of the present invention is stored.

On the one hand, the recording medium can be any entity or device capable of storing the program, such as at least one volatile and/or non-volatile memory. For example, the medium can include a storage medium, such as a rewritable non-volatile memory, a ROM, a CD-ROM or a microelectronic circuit type ROM, or a magnetic recording medium or a hard disk. This memory can, for example, comprise a graphics card (or video card) memory, this type of memory being in particular configured to process image data (or video data).

On the other hand, this recording medium can also be a transmissible medium such as an electrical or optical signal, such a signal being able to be conveyed via an electrical or optical cable, via conventional or Hertzian radio or by self-directed laser beam or by other means. The computer program according to the present invention can in particular be downloaded using a wired or wireless network, local or otherwise (e.g. Bluetooth®, Wi-Fi, Ethernet, internet, 4G, 5G or others).

Alternatively, the recording medium can be an integrated circuit in which the computer program is incorporated, the integrated circuit being able to execute or be used in the execution of the method under discussion.

According to another aspect, the present invention relates to a device for processing (or a device for controlling) an electrical signal for an ultrasonic transmitting device, this device being configured to carry out the method of the first aspect of the present invention.

According to one example, the processing device comprises a memory associated with a processor, this memory comprising a computer program according to the present invention.

According to one example, the present invention relates to a device for processing an electrical signal for an ultrasonic transmitting device, said processing device comprising:
a generation module configured to generate an electrical signal defining ultrasonic waves, the periodicity of the electrical signal being modified by modulation of the phase of the signal; and
a supply module configured to supply the electrical signal to the ultrasonic transmitting device to cause the ultrasonic waves to be emitted.

The processing device can have functions that correspond to the steps (or operations) of the method according to the present invention. In particular, the various embodiments mentioned in the present invention in relation to the method of the present invention as well as the associated advantages can be applied in a similar manner to the processing device (and vice versa).

The features and advantages of the invention will become apparent more clearly upon reading the description below, provided purely by non-limiting way of example, and with reference to the appended figures. In particular, the examples shown in the figures can be combined with one another unless there are clear inconsistencies.

### BRIEF DESCRIPTION OF THE FIGURES

Other features and advantages of the present invention will become apparent from the description of the non-limiting exemplary embodiments of the present invention set out below with reference to the appended FIGS. 1 to 14, in which:

FIG. 1 schematically shows an electrical signal from the prior art, without phase modulation, for controlling an ultrasonic transmitting device according to one example;

FIG. 2 schematically shows the frequency spectrum of the electrical signal shown in FIG. 1 without phase modulation according to one example:

FIG. 3 schematically shows an ultrasound system, comprising a processing device and an ultrasonic transmitting device, according to exemplary embodiments of the present invention;

FIG. 4 schematically shows the ultrasound system shown in FIG. 3, according to exemplary embodiments of the present invention;

FIG. 5 schematically shows the processing device shown in FIG. 3, according to exemplary embodiments of the present invention;

FIG. 6 schematically shows, in the form of a chart, the steps of a processing method carried out by a processing device, according to exemplary embodiments of the present invention;

FIG. 7 schematically shows the modulation of an electrical signal during the processing method shown in FIG. 6, according to exemplary embodiments of the present invention;

FIG. 8 schematically shows the frequency spectrum of the electrical signal shown in FIG. 7, according to exemplary embodiments of the present invention;

FIG. 9 schematically shows the modulation of an electrical signal during the processing method shown in FIG. 6, according to exemplary embodiments of the present invention;

FIG. 10 schematically shows the frequency spectrum of the electrical signal shown in FIG. 9, according to exemplary embodiments of the present invention;

FIG. 11 schematically shows the modulation of an electrical signal during the processing method shown in FIG. 6, according to exemplary embodiments of the present invention;

FIG. 12 schematically shows the frequency spectrum of the electrical signal shown in FIG. 11, according to exemplary embodiments of the present invention;

FIG. 13 schematically shows the modulation of an electrical signal during the processing method shown in FIG. 6, according to exemplary embodiments of the present invention; and

FIG. 14 schematically shows the frequency spectrum of the electrical signal shown in FIG. 13, according to exemplary embodiments of the present invention.

### DESCRIPTION OF THE EMBODIMENTS

The present invention relates to methods and devices for processing (or controlling) an electrical signal for an ultrasonic transmitting device.

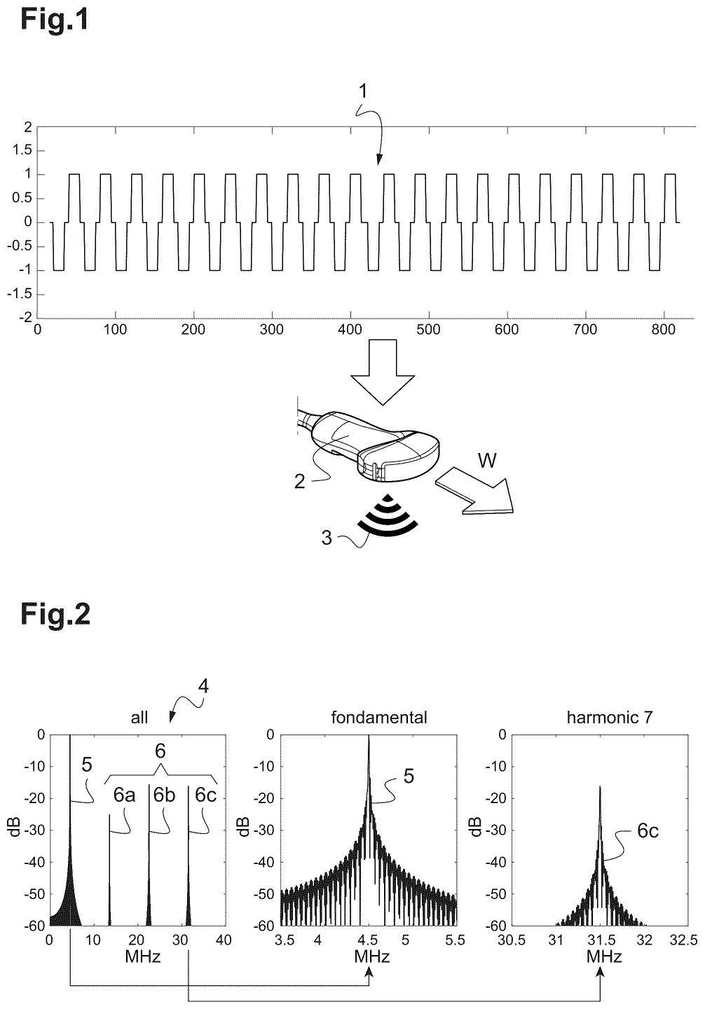

As shown in FIG. 1 in one example, an ultrasonic transmitting device 2 is configured to emit ultrasonic waves W in response to one or more electrical signals 1. To do this, a control system (not shown) can be configured to supply these electrical signals 1 to the ultrasonic transmitting device 2. This control system can in particular comprise one or more electronic pulsers (known simply as pulsers) or linear amplifiers, configured to generate the electrical signal(s) 1 intended to control the ultrasonic transmitting device 2.

The ultrasonic transmitting device 2 can comprise one or more ultrasonic transducers, each of them being controlled by an electrical signal 1 supplied by the control system. These electrical signals 1 can be transmitted via a transmission chain from the pulsers to the transducers of the transmitting device 1.

It has been noticed that the generation of ultrasonic waves W by such an ultrasonic transmitting device 2 creates electromagnetic (EM) emissions 3 that might need to be minimised, for example in their radiation, directions and intensity. In certain cases, it is desirable to limit the EM radiation 3 of such a transmitting device whilst at the same time ensuring that it performs well, for example in terms of spectral response and/or acoustic energy (or power) supplied. It can in particular be desirable to limit the level of EM emissions 3 with minimal impact on the desired operating performance of the ultrasonic transmitting device 2.

As shown in FIG. 2, the frequency spectrum 4 of the electrical signal 1 supplied to the ultrasonic transmitting device 2 generally comprises at least one main peak at a fundamental frequency 5 (supplying a substantial amount of electrical energy) and secondary peaks 6 at harmonic frequencies, i.e. peaks 6a, 6b and 6c in this example. These harmonic frequencies correspond to multiples of the fundamental frequency. The generation of such harmonics results from the periodic nature of the electrical wave signal 1.

These harmonics 6 are outside of the bandwidth of the transducers of the ultrasonic transmitting device 2 and are not converted into acoustic energy but dissipated in the form of heat, which limits the energy efficiency of the device. The emission of such harmonics can also lead to excessive electromagnetic radiation, which can be problematic in particular when a maximum limit of this radiation has to be observed (for example in compliance with a regulation imposed to obtain certification of the system).

In order to limit the harmonics and prevent excessive electromagnetic radiation, it is known to use linear pulsers, or amplifiers, that are able to generate sinusoidal signals, but this technique is particularly expensive and not very effective, particularly in the medical field, in particular because of the fact that the waves generated in this way are limited in power and produce a lot of heat.

The aforementioned constraints and/or problems therefore require compromises to be made or certain features of such an ultrasonic transmitting device to be limited in order to guarantee good performance and acceptable or even optimum ageing depending on market requirements for the system as a whole. These constraints and/or problems can also make it difficult to certify such a wave-emitting device to current standards.

The present invention aims to address the aforementioned problems and constraints by processing the electrical signal supplied to an ultrasonic transmitting device in order to limit the level of harmonics contained in the electrical signal, while preserving the desired maximum acoustic energy (or signal level) at the fundamental frequency, so as to guarantee that the ultrasonic transmitting device performs well.

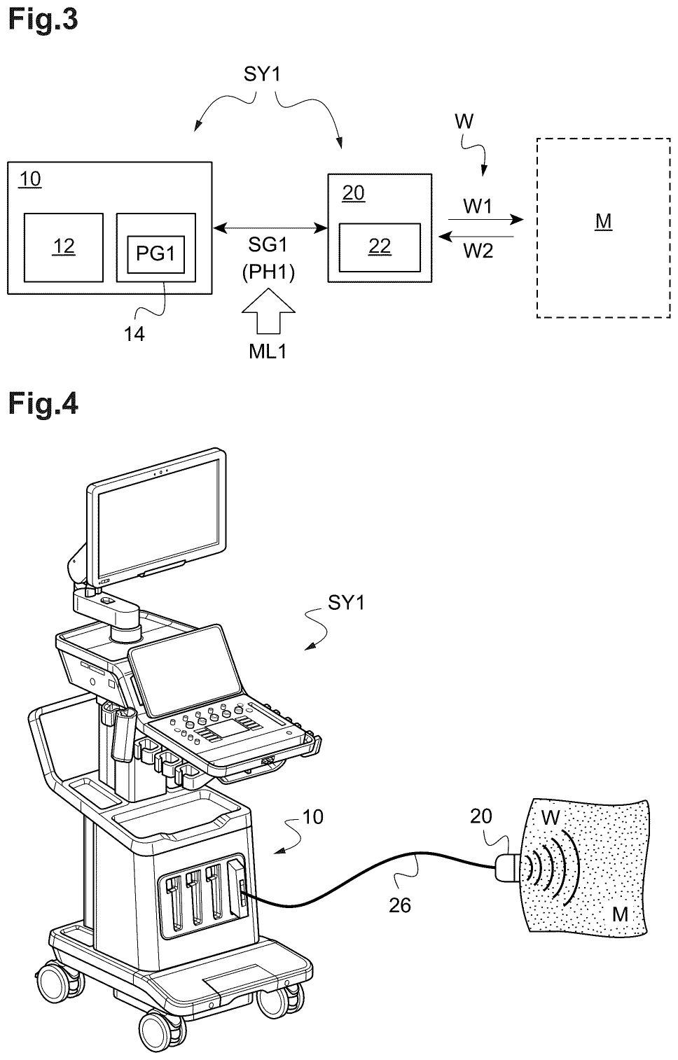

Methods and devices for processing (or controlling) an electrical signal for an ultrasonic transmitting device will now be described according to specific embodiments of the invention with joint reference to FIGS. 3 to 14. Unless stated otherwise, common or similar elements in several figures bear the same reference numerals and have identical or similar features such that these common elements are not generally described again for the sake of concision.

The terms “first” (or “first, second”, etc.) are used in this document by arbitrary convention to enable different elements (such as operations, devices, etc.) carried out or used in the embodiments described below to be identified or distinguished.

As set out above, the present invention relates in particular to a processing method carried out by an ultrasonic transmitting device, as well as such a device. FIG. 3 schematically shows an ultrasound system SY1 comprising a processing device (or control device) 10 and an ultrasonic transmitting device 20 (also known as an ultrasonic transmission device, or device for emitting ultrasonic waves, or ultrasonic transducer device) according to exemplary embodiments of the present invention.

More specifically, the processing device 10 is configured to control the ultrasonic transmitting device 20. To this end, the processing device 10 generates one or more electrical signals SG1 that it transmits to the ultrasonic device 20 to cause ultrasonic waves W1 to be emitted. For the purpose of simplifying the description of the present invention, it will hereinafter be considered that the processing device 10 generates and transmits an electrical signal SG1 to the ultrasonic device 20, it being understood that this electrical signal SG1 can comprise a plurality of electrical signals. The electrical signal SG1 emitted in this way controls the ultrasonic waves W1 emitted by the transmitting device 20.

The ultrasonic transmitting device 20, also referred to hereinafter as the ultrasonic device, is configured to emit, and potentially also receive, waves W2.

This ultrasonic device 20 can, for example, take the form of an ultrasonic probe (for example an echographic probe). The nature of these ultrasonic waves W depends on the configuration of the ultrasonic device 20, particularly in view of how it is used.

According to one example, the processing device 10 and the ultrasonic device 20 are separate devices. Alternatively, all or part of the processing device 10 can be implemented in the ultrasonic device 20.

Generally speaking, the ultrasound system SY1, and more specifically the processing device 10 and the ultrasonic transmitting device 20, can be stationary or mobile.

According to one example, the processing device 10 and the ultrasonic device 20 form a single device, which is stationary or mobile as the case may be.

For example, the ultrasonic device 20 can be connected to the processing device 10 by a cable or can communicate wirelessly with it. In the latter case, the ultrasonic device 20 can, for example, comprise a battery and receive communication signals from the processing device 10, these signals representing the electrical signal SG1 (for example the control frequencies and/or any information included in the electrical signal). The ultrasonic device 20 can then reproduce the electrical signal SG1 internally based on the communication signals received.

The ultrasonic device 20 can, for example, be a conventional ultrasound wave emitting device, this device being controlled by means of the processing device 10 according to a processing method in accordance with the present invention.

As shown in FIG. 3, the processing device 10 comprises a processor 12 and a memory 14 in this example. The processing device 10 is configured to control the ultrasonic device 20 by generating and sending an electrical signal SG1. Once it has been generated, this electrical signal SG1 is thus transmitted to the ultrasonic device 20 to cause said ultrasonic device 20 to emit ultrasonic waves W1 in the direction of and/or into a medium M. The electrical signal SG1 generated in this way is representative of (or defines) the ultrasonic waves W1 projected into the medium M. To this end, the processing unit 10 can, for example, be or comprise at least one electronic pulser, also known as a “pulser” able to generate the electrical signal.

The electrical signal SG1 is a wave (or alternating) signal which is processed by the processing device 10 to control the ultrasonic device 20 so as to obtain good performance of the ultrasonic device 20 whilst at the same time minimising its EM emissions. As described below, the processing device 10 is configured to modify the periodicity of the electrical signal SG1 by modulation ML1 of the phase PH1 (FIG. 3) of the electrical signal SG1. The processing device 10 can also perform various processing operations when the electrical signal SG1 is generated (amplification, filtering, digitising, conditioning of the signal, etc.).

In some examples, the processor 12 and the memory 13 can be incorporated in the processing device 10 shown in FIG. 3 or can be incorporated in a computer or a computing device communicatively connected thereto.

The memory 14 can store instructions defining the steps of the methods described in the present invention in the form of a computer program PG1. In this respect, the memory 14 constitutes a recording medium (or storage medium) in accordance with specific embodiments, readable by the processing device 10, and on which a computer program PG1 according to specific embodiments is stored. This computer program PG1 includes instructions for carrying out the steps of a processing method, specific embodiments of which are described in the present invention. The processor 12 is thus configured to carry out the instructions of the computer program PG1 in order to carry out the steps of the processing method.

Depending on the configuration and type of computing device under consideration, the memory 14 can be volatile (such as RAM), non-volatile (such as ROM, Flash, EEPROM, etc. or any other storage device and/or computer-readable medium as described below) or a combination of both. The memory 14 can, for example, be managed in DMA (Direct Memory Access) mode. The memory 14 used by the processing unit 10 can, for example, comprise all or part of a graphics card (or video card) memory, this type of memory being in particular configured to process and/or send image data that can be used to display one or more images on a display screen (or unit).

The processing device 10 can take the form of various appropriate computing means (such as a workstation, computer, server, etc.) comprising all or some of the elements described above and possibly other elements that are not mentioned.

As shown in FIG. 3, the ultrasonic device 20 can comprise, for example, one or more transducers (also known as transducer elements) 22, each being configured to convert an electrical signal SG1 supplied by the processing device 10 into ultrasonic waves W (and possibly also vice versa). The transducers 22 can thus be configured to emit waves (or ultrasonic pulses, or ultrasonic beams) W1 into the medium M, which corresponds to an emission operation. These transducer elements can be arranged in any way, for example in a line of transducers, a matrix, an array of transducers or any other suitable configuration.

It should be noted that the transducers 22 can potentially also be configured to receive ultrasonic signals W2 from the medium M in a receiving operation, for example in response to the transmitted waves W1, although variants where the ultrasonic device 20 only functions in transmission mode are also possible.

The ultrasound system SY1 (FIG. 3) can be an ultrasound imaging system, for example in the medical field. The ultrasound images generated by the system can either be analysed in real-time, for example by a computer or an algorithm, and/or an artificial intelligence module, or analysed later on and/or at a location other than the one where the system SY1 is located.

The system SY1 can be a medical ultrasound system. Similarly, the transmitting device 20 can be a medical ultrasonic probe.

For example, the system SY1 can be associated with an ultrasonic probe 20, in order to study a medium M (FIG. 3), in particular to collect ultrasound data from such a medium M. The medium M observed in this way can take various forms depending on the case. It can, for example, be metal, or tissue of a living being, in particular human or animal tissue. It is also possible to observe a medium M comprising one or more mineral structures, for example (gravel, volcano, soil mapping, for example seabeds, etc.).

The ultrasound system SY1 can be designed for a variety of applications, in particular in the fields of acoustics, material studies, medical imaging and/or biomedicine.

FIG. 4 schematically shows a non-limiting exemplary embodiment of the system SY1 as described above with reference to FIG. 3, i.e. in this case an ultrasound imaging system. In this example, the processing unit 10 is included in the body of a control station, which is also equipped with a control interface and a display device. The ultrasonic device 20 takes the form of an ultrasonic probe, communicatively connected in this example to the processing device 10 via a connection cable 26.

The ultrasound system SY1 can be configured to produce various types of ultrasound images, for example a B-mode image of the medium M (B-mode greyscale image), a so-called Doppler image showing the movement of fluid in the observed medium, and/or an image showing a mechanical property of the medium (for example ShearWave™ Elastography image data).

According to one example, the electrical signal SG1 transmitted by the processing device 10 thus causes the ultrasonic device 20 to emit ultrasound compression waves generating shear waves in a medium M.

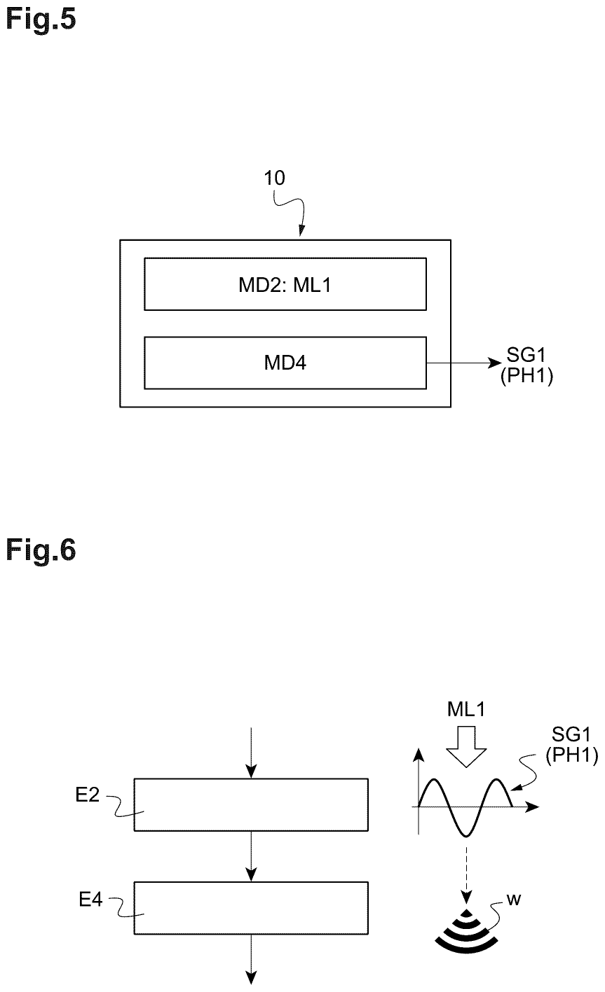

As shown in FIG. 5 according to one embodiment, the processor 12 of the processing device 10, controlled by the computer program PG1 (FIG. 3), can use a generation module MD2 and a supply module MD4.

More specifically, the generation module MD2 can be configured to generate an electrical signal SG1 defining ultrasonic waves W, the periodicity of the electrical signal SG1 being modified (or adapted) by modulation ML1 of the phase PH1 of the signal SG1.

The supply module (or transmission module) MD4 can be configured to supply the electrical signal SG1 to the ultrasonic transmitting device 20 to cause the ultrasonic waves W to be emitted.

The configuration and operation of the modules MD2-MD4 of the processing device 10 will become apparent more clearly in the exemplary embodiments described below with reference to the figures. The modules MD2-MD4 as shown in FIG. 5 only represent one non-limiting exemplary embodiment of the invention.

Generally speaking, for each step in the processing method of the present invention, the processing device of the invention can comprise a corresponding module configured to carry out said step (and vice versa).

Embodiments of the processing method of the invention are now described with reference to FIGS. 6 to 14. In these examples, the processing method is carried out by the ultrasound system SY1 (FIGS. 3, 4 and 5), and more specifically by the processing device 10. To do this, the processing device 10, cooperating with the ultrasonic device 20, can run the computer program PG1.

During a processing step E2 (FIG. 6), the processing device 10 generates an electrical signal SG1 defining ultrasonic waves W, the periodicity of the electrical signal SG1 being modified, adapted or controlled by modulation ML1 of the phase PH1 of the signal SG1.

The electrical signal SG1 is a wave (or alternating) signal intended to control the ultrasonic device 20. The phase PH1 (or periodicity) of the signal SG1 is disturbed by modulation ML1. This modulation PH1 can have the effect that the electrical signal SG1 is not totally periodic or has a modified periodicity compared with a theoretical case where such modulation ML1 would not be applied to the signal SG1.

According to one example, the phase PH1 is the instantaneous phase. As a person skilled in the art understands, the instantaneous phase of the electrical signal SG1 is different from the original phase. For example, for a sinusoidal signal SG1, this signal can be defined as follows

SG1=sin(2·rr·fO·t+φ) [Math. 1]
where φ is the original phase and where (2*π*f0*t+φ) is the instantaneous phase at point t.

Modulation ML1, which can be performed in various ways as described below in examples, can in particular be configured to cause the frequency spectrum of the electrical signal SG1 (and thus the ultrasonic waves W emitted) to broaden. This broadening can reduce the frequency harmonics contained in the electrical signal SG1 and thus reduce the EM emissions of the ultrasonic device 20, or more generally of the ultrasound system SY1.

During a supply or transmission step E4 (FIG. 6), the processing device 10 supplies the electrical signal SG1 to the ultrasonic device 20 to cause the ultrasonic waves W1 to be emitted

The electrical signal SG1 can comprise periodic cycles. The waveform of the signal can vary from case to case. The number of cycles during which the electrical signal SG1 is transmitted can also be adapted as required.

According to one example, the electrical signal SG1 is transmitted for at least several tens of cycles of the signal, so as to cause a continuous emission of ultrasonic waves W1 towards a medium M (for example during an emission or “push” lasting approximately 100 μs) The ultrasonic waves W1 emitted in this way are, for example, compression waves generating shear waves in the medium M under consideration (in a shear wave elastography mode). By its very nature, the continuous emission of this type of wave tends to generate significant EM radiation, which might need to be prevented.

According to one example, modulation ML1 of the phase PH1 of the electrical signal SG1 is deterministic over time. In other words, modulation PH1 is performed according to a predefined configuration. In particular, the cycle or cycles of the signal SG1 in which the phase PH1 is modulated can be selected in a deterministic or predefined manner.

According to one example, modulation ML1 of the phase PH1 of the electrical signal SG1 is random over time. In particular, the cycle or cycles of the signal SG1 in which the phase PH1 is modulated can be selected randomly.

The present invention advantageously makes it possible to limit the EM radiation emitted by the ultrasonic transmitting device 20 whilst at the same time ensuring that it performs adequately, for example in terms of spectral response and/or acoustic energy (or power) supplied. It is thus advantageously possible to limit the level of EM emissions with minimal impact (ideally no impact) on the desired operating performance of the ultrasonic transmitting device 20.

To do this, the electrical signal supplied to the ultrasonic transmitting device 20 is adapted (or processed) by modulation of the phase PH1 of the signal SG1. By disturbing the phase PH1 of the electrical signal SG1, it is advantageously possible to reduce the harmonics contained in this signal and thus minimise EM radiation. This modulation can advantageously be carried out in an existing transmitting system or device without the need for additional devices and/or structural modifications to the existing system or device. The complexity and costs involved in carrying out the method are therefore limited.

In particular, it is advantageously possible to develop an efficient ultrasonic transmitting device that enables optimum use and at the same time generates minimum electromagnetic emissions during operation and has limited complexity in terms of design, manufacturing and use.

Electromagnetic emissions can be limited in various operating modes of the ultrasonic transmitting device. Examples of different operating modes of a transmitting device in the form of an ultrasonic probe can comprise a B-mode (i.e. brightness mode), a Doppler mode or a Shear-Wave mode (i.e. shear wave elastography mode). In other words, it is not necessary to change or modify the specific operating mode of an ultrasonic transmitting device to obtain reductions in EM emissions, this being possible independently of the selection of the operating mode of the device.

For reference, FIGS. 1 and 2 show a reference electrical signal 1 without modulation ML1, equivalent to the electrical signal SG1 that would theoretically be generated in the examples of the present invention if no phase shift ML1 were applied in accordance with the present invention.

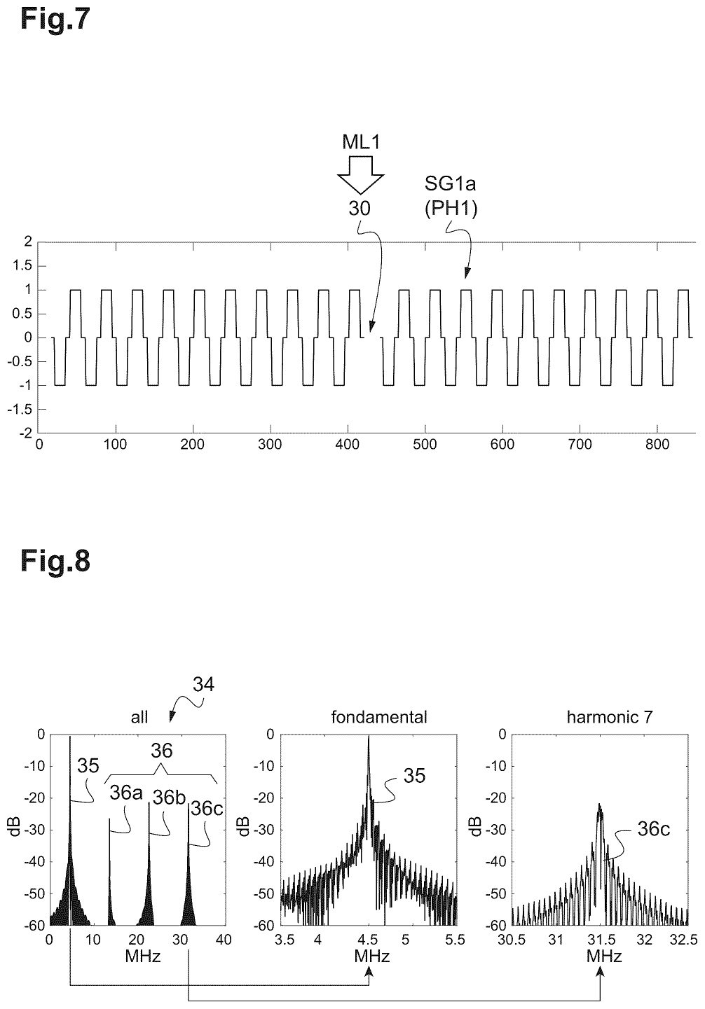

Exemplary embodiments of the modulation ML1 of the electrical signal SG1 carried out by the processing device 10 during the generation step E2 of the processing method are now described with reference to FIGS. 7 to 14.

It should be noted that it is possible to modulate ML1 the electrical signal SG1 according to various embodiments, such as those described below, this modulation ML1 being able to combine at least any two embodiments among those described below with reference in particular to FIGS. 7-14. In other words, it is possible to cumulatively apply a plurality of the modulation techniques described below in examples.

According to a first embodiment shown in FIGS. 7 and 8, the phase PH1 of the electrical signal SG1, in this case SG1a, is modulated ML1 (step E2, FIG. 6) by introducing (or adding), into the electrical signal SG1a, at least one delay (or wait period) 30, corresponding to a period during which the voltage of the electrical signal SG1a is kept at a constant value. By way of example, this constant value is fixed at zero (0 Volts), although other values are possible.

In this example, FIG. 7 shows the modulated electrical signal SG1a, where time is shown along the x-axis (in number of clock cycles) and the voltage of the signal is shown along the y-axis. As shown, a single delay 30 is introduced into the electrical signal SG1 in this example, although it is possible to introduce a plurality.

The introduction of one or more time delays 30 causes a division into N ultrasonic sub-emissions, which are shorter than if no time delay were applied, N being an integer at least equal to 1. This produces a discontinuous periodic electrical signal SG1a. In other words, adding the one or more delays 30 disrupts the periodicity of the electrical signal SG1a.

According to one example, N is equal to 2 or more, which corresponds to the introduction of a plurality of delays 30 into the electrical signal SG1a. The addition of a delay 30 between the ultrasonic sub-emissions can advantageously minimise the harmonics contained in the electrical signal SG1a (and therefore in the outgoing ultrasonic waves W) and thus limit the EM radiation caused by these harmonics.

According to one example, modulation ML1 is performed such that N is at least equal to 4 (N≥4). It is thus possible to introduce delays 30 between ultrasonic sub-emissions collectively forming the electrical signal SG1a. The repetition of these delays 30 advantageously makes it possible to produce an electrical signal SG1a, and therefore an ultrasonic wave W, over a relatively long period whilst at the same time effectively minimising electromagnetic radiation. In particular, the repetition of delays 30 over time (for example 10, 20, 30 or 40 delays 30) makes it possible to broaden the spectrum of the signal and therefore cause a redistribution of the electrical energy, which leads to a reduction in the peak spectral density of electromagnetic emissions.

By way of example, compression waves can advantageously be generated in the form of continuous ultrasonic emissions, known as “push”, i.e. cycles of electrical signals SG1 which are interspersed with delays 30 (for example 4 or more delays) and enable shear waves to be created in the medium of interest while limiting the electromagnetic radiation likely to be emitted by the probe 20.

According to one example, the length of the delay or delays 30 introduced into the electrical signal SG1 is strictly less than a half period (or a third or a quarter of the period) of the electrical signal SG1. It is thus possible to minimise wait times during delays 30, making it possible to speed up the generation of ultrasonic waves W, while limiting electromagnetic radiation by effectively disrupting the periodicity of the signal. This embodiment is in particular configured to generate shear waves, which requires the generation of a relatively long “push” of the electrical signal SG1. It therefore becomes essential to limit the length of the delays 30 as much as possible to enable the method to be carried out in a limited amount of time.

According to one specific example, the electrical signal SG1 generated in E2 (FIG. 6) comprises a plurality of successive sequences, each sequence comprising at least one wave cycle (or at least one period) of the electrical signal SG1 according to a given frequency, a delay 30 being applied between (or separating) each pair of consecutive sequences. According to one example, the electrical signal SG1 generated during all these sequences over time is in phase (or is identical), which advantageously makes it possible to limit the disturbances or interference that can result between the different sequences of the electrical signal SG1. According to one example, the frequency of the electrical signal SG1 in each sequence is identical (in other words, the frequency is constant outside of the delay periods), which advantageously makes it possible in particular to use a transducer with a very narrow frequency bandwidth or to repeat the same waveform offset in time to reduce the memory space required to store the waveforms emitted.

More specifically, as shown in FIG. 8, the frequency spectrum 34 of the electrical signal SG1a comprises, for example, a main peak 35 at a fundamental frequency and secondary peaks 36a, 36b and 36c (collectively referenced 36) at respective harmonic frequencies. Modulation ML1 by adding a delay 30 in this example makes it possible to substantially reduce the harmonics 36, in particular the harmonic 36c (multiple of 7 of the fundamental frequency), whilst maintaining the peak 35 at the fundamental frequency at a high level. In particular, the reduction in harmonics 36 results from spectral broadening caused by the time delay 30 which disrupts the periodicity of the signal SG1a.

According to one example, the delay or delays 30 are configured to be less than the period of the electrical signal SG1a, i.e. less than the length of a periodic cycle of the signal SG1a. In this way, it is advantageously possible to maintain the density of electrical energy supplied by the signal SG1 despite the application of modulation ML1.

By way of example, the electrical signal SG1a modulated in this way produces a continuous ultrasonic emission (or “push”) of approximately 100 μs with a period of 0.5 μs. The delay or delays 30 are, for example, around 0.2 μs.

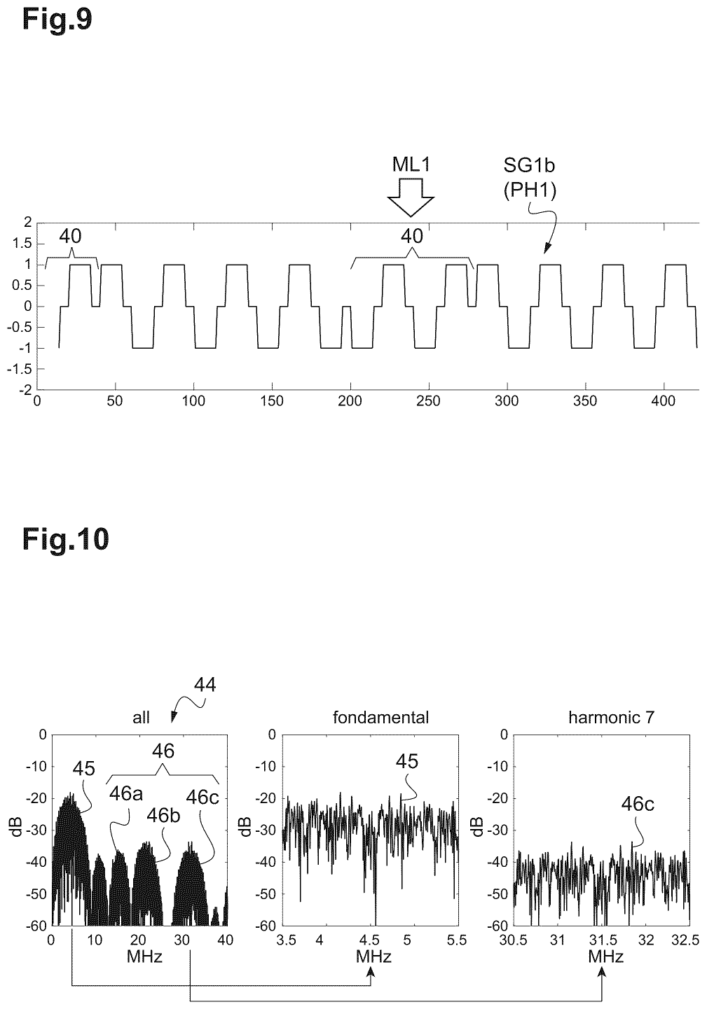

According to a second embodiment shown in FIGS. 9 and 10, the phase PH1 of the electrical signal SG1, in this case SG1b, is modulated ML1 (step E2, FIG. 6) by applying (or introducing) an angular shift of the phase PH1 of the electrical signal SG1B in at least one cycle of said signal. This angular shift 40 corresponds to a phase shift of the signal SG1 by a given angular value. The angular value of this phase shift can be adapted if required. By way of example, this angular value can be fixed at 30° or 40°, although other values are possible.

According to one example, an angular shift 40 is applied to a single electrical signal SG1a cycle. According to another example, such an angular shift 40 is applied to a plurality of electrical signal SG1b cycles. The shift 40 can have the same value or a different value from one phase-shifted cycle to another.

In this example, FIG. 9 shows the modulated electrical signal SG1b, where time is shown along the x-axis (in number of clock cycles) and the voltage of the signal is shown along the y-axis. As shown in this example, an angular shift 40 of a value of 180° is applied to the electrical signal SG1 in certain cycles with respect to other cycles where the signal is not phase-shifted.

The application of one or more angular shifts 40 results in a phase shift between the cycles of the electrical signal SG1b such that at least one cycle of the signal is phase-shifted relative to at least another cycle of the signal. In this case, this results in a continuous periodic signal, the periodicity of which is disturbed or modified by the angular shift or shifts 40.

In the example shown in FIG. 9, the angular shift of the phase PH1 is fixed at a value of 180°, causing a sign reversal of the electrical signal in the phase-shifted cycle or cycles.

The application of one or more angular shifts can advantageously minimise the harmonics contained in the electrical signal SG1b (and therefore in the outgoing ultrasonic waves W) and thus limit the EM radiation caused by these harmonics.

More specifically, as shown in FIG. 10, the frequency spectrum 44 of the electrical signal SG1b comprises, for example, a main peak 45 at a fundamental frequency and secondary peaks 46a, 46b and 46c (collectively referenced 46) at respective harmonic frequencies. Modulation ML1 by angular shift in this example makes it possible to substantially reduce the harmonics 46, in particular the harmonic 46c (multiple of 7 of the fundamental frequency), whilst maintaining the peak 45 at the fundamental frequency at a high level.

In particular, the reduction in harmonics 46 results from spectral broadening caused by the angular shift of the phase which disrupts the periodicity of the signal SG1b. As shown, this results in a multiplication of the frequency peaks contained in the signal SG1b compared to if no modulation ML1 had been applied, which results in a redistribution of the electrical energy in the frequency spectrum of the signal SG1b.

According to one example, an angular shift 40 of 180° is randomly applied to the electrical signal SG1b to cause an inversion of the signal SG1b in at least one randomly chosen cycle. Such a random angular shift can also be applied for a non-zero value other than 180°.

According to one example, an angular shift 40 of 180° is deterministically applied to the electrical signal SG1b to cause an inversion of the signal SG1b in at least one cycle chosen in a predefined manner. Such a deterministic angular shift can also be applied for a non-zero value other than 180°.

According to one example, angular shifts 40 with different values are respectively applied to a plurality of cycles of the electrical signal SG1b.

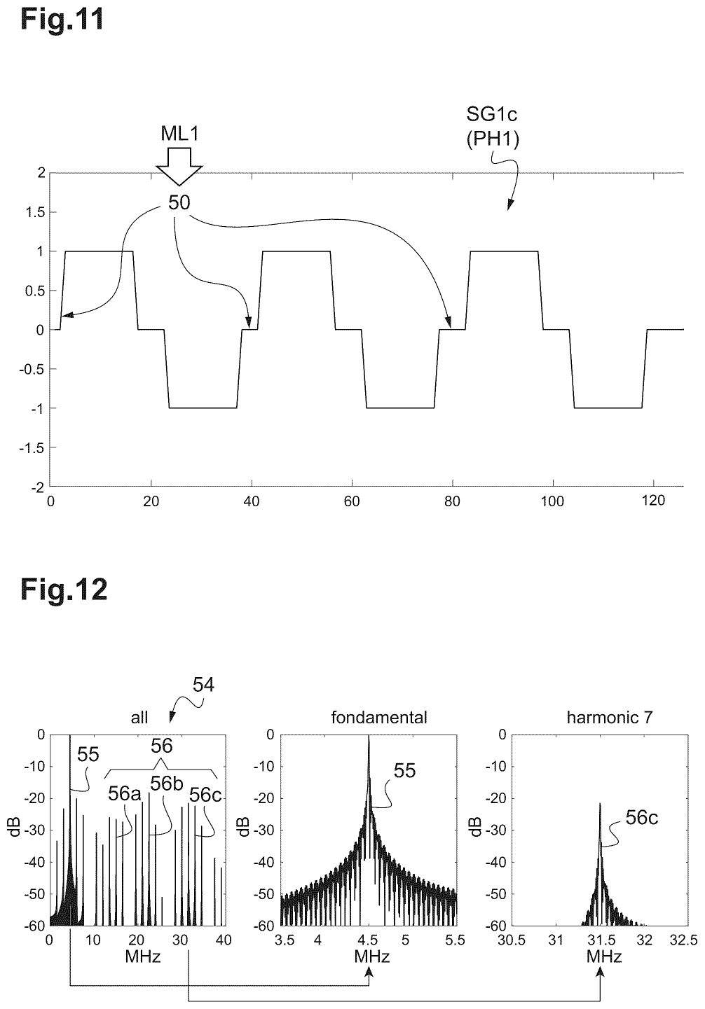

FIGS. 11 and 12 show an example similar to the one shown in FIGS. 9-10, in which the phase PH1 of the electrical signal SG1, in this case SG1c, is modulated ML1 (step E2, FIG. 6) by applying angular shifts, referenced 50 (FIG. 11), of 18°, 35° and 53° of the phase PH1 of the electrical signal SG1. This is equivalent to applying phase shifts to the signal SG1c according to these angular values.

More specifically, as shown in FIG. 12, the frequency spectrum 54 of the electrical signal SG1c comprises, for example, a main peak 55 at a fundamental frequency and secondary peaks 56a, 56b and 56c (collectively referenced 56) at respective harmonic frequencies. Modulation ML1 by angular shift in this example makes it possible to substantially reduce the harmonics 56, in particular the harmonic 56c (multiple of 7 of the fundamental frequency), whilst maintaining the peak 55 at the fundamental frequency at a high level.

The reduction in harmonics 56 results from spectral broadening caused by the angular shift of the phase which disrupts the periodicity of the signal SG1c. As shown, this results in a multiplication of the frequency peaks contained in the signal SG1c compared to if no modulation ML1 had been applied, which results in a redistribution of the electrical energy in the frequency spectrum of the signal SG1c.

According to one example, the phase PH1 of the electrical signal SG1 is modulated ML1 (step E2, FIG. 6) by applying (or introducing) an angular shift 50 to each half period of the signal SG1. This angular offset 50 can be the same value for each half-period, or vary as required. By applying two angular shifts 50 per period in the electrical signal SG1c, it is possible to effectively broaden the frequency spectrum of this signal and thus minimise the peak of electromagnetic emissions while maintaining a high level of electrical energy supplied by the electrical signal SG1. According to one example, the angular shifts 50 introduced into the signal SG1 increase, or decrease, gradually over time. By way of example, the angular shift introduced at each half period of the signal SG1 increases, or decreases, gradually over time. It is thus possible to form, for example, waves that are increasingly close together, or far apart, over time, which makes it advantageously possible to efficiently distribute the electrical energy of the electrical signal SG1 (spectral broadening), and thus to minimise electromagnetic emissions. A combination of increasing and decreasing angular shift phases is also possible. Thus, according to one example, the electrical signal SG1 comprises at least one first period (or first phase) during which the angular shift introduced into the electrical signal increases gradually, and at least one second period (or second phase) during which the angular shift introduced into the electrical signal decreases gradually. The electrical signal SG1 can in particular comprise alternating first and second phases in which these angular shifts increase and decrease respectively over time.

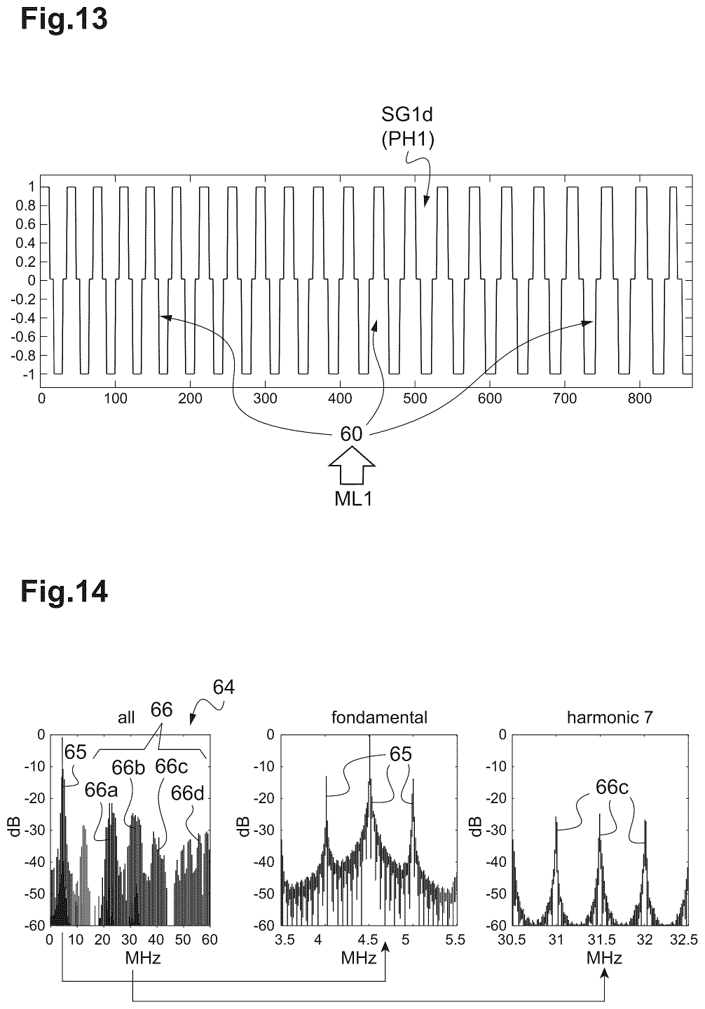

According to a fourth embodiment shown in FIGS. 13 and 14, the phase PH1 of the electrical signal SG1, in this case SG1d, is modulated ML1 (step E2, FIG. 6) by a variation 60 over time of the period of the cycles of the electrical signal SG1d.

This variation 60 of the period can thus comprise, for example, an increase and/or a decrease in the period of the electrical signal SG1c over time. The way in which the period of the signal SG1d varies over time can be adapted if required.

According to one example, a variation 60 of the period is applied continuously to a plurality of consecutive cycles of the electrical signal SG1d. This variation can, for example, be linear, or non-linear, over time.

In this example, FIG. 13 shows the modulated electrical signal SG1d, where time is shown along the x-axis (in number of clock cycles) and the voltage of the signal is shown along the y-axis. As shown in this example, the period of the electrical signal SG1d is increased gradually over time over a plurality of cycles.

The application of such a variation 60 can advantageously minimise the harmonics contained in the electrical signal SG1d (and therefore in the outgoing ultrasonic waves W) and thus limit the EM radiation caused by these harmonics.

More specifically, as shown in FIG. 14, the frequency spectrum 64 of the electrical signal SG1d comprises, for example, a main peak 65 at a fundamental frequency and secondary peaks 66a, 66b, 66c and 66d (collectively referenced 66) at respective harmonic frequencies. Modulation ML1 by variation of the period in this example makes it possible to substantially reduce the harmonics 66, in particular the harmonic 66b (multiple of 7 of the fundamental frequency), whilst maintaining the peak 65 at the fundamental frequency at a high level.

In particular, the reduction in harmonics 66 results from spectral broadening caused by the variation 40 of the period which disrupts the periodicity of the signal SG1d. As shown, this results in a multiplication of the frequency peaks contained in the signal SG1d compared to if no modulation ML1 had been applied, which results in a redistribution of the electrical energy in the frequency spectrum of the signal SG1b.

Thus, the modulation ML1 applied during step E2 (FIG. 6) can therefore be performed in a variety of ways, the embodiments above only being described by way of example.

According to one example, modulation ML1 (step E2, FIG. 6) of the phase PH1 is configured so that the electrical signal SG1 supplies electrical energy E2 at least equal to theoretical energy E1 that would be supplied by the electrical signal SG1 if its phase PH1 were not disturbed by said phase modulation ML1 (E2>E1). By respecting this principle of maintaining electrical energy, it is advantageously possible to obtain satisfactory performance, particularly in terms of the acoustic energy (or power) supplied at the output of the ultrasonic device 20. It is thus possible to advantageously limit the level of electromagnetic emissions with minimal impact (ideally no impact) on the desired operating performance of the ultrasonic device 20.

According to one example, the phase is modulated ML1 (step E2, FIG. 6) while maintaining the same frequency f on each cycle of the electrical signal SG1.

As a person skilled in the art understands, all of the embodiments and variants described above, some of which have been deliberately simplified to make them easier to explain, only constitute non-limiting exemplary embodiments of the present invention. In particular, a person skilled in the art could envisage adapting or combining the embodiments and variants described above in order to address a specific need.

The present invention is therefore not limited to the exemplary embodiments described above but extends in particular to a processing method that would include secondary steps without departing from the scope of the present invention. The same would apply to a processing system for carrying out such a method.

1. Method for processing an electrical signal (SG1) for an ultrasonic transmitting device (20), said method comprising:
generating (E2) an electrical signal (SG1) defining ultrasonic waves (W), the periodicity of the electrical signal being modified by modulation (ML1) of the phase (PH1) of the signal; and
supplying (E4) the electrical signal (SG1) to the ultrasonic transmitting device (20) to cause the ultrasonic waves (W) to be emitted.

2. Method according to claim 1, wherein the modulation of the phase of the signal is configured to cause a broadening of the frequency spectrum of the electrical signal.

3. Method according to claim 1 or 2, wherein the modulation of the phase of the electrical signal is deterministic over time.

4. Method according to claim 1 or 2, wherein the modulation of the phase of the electrical signal is random over time.

5. Method according to any one of the preceding claims, wherein modulation (ML1) of the phase (PH1) of the electrical signal comprises:
introducing, into the electrical signal (SG1a), at least one delay (30) corresponding to a period during which the voltage of the electrical signal is kept at a constant value.

6. Method according to claim 5, wherein said at least one delay (30) is less than the period of the electrical signal.

7. Method according to one of claims 1 to 4, wherein modulation (ML1) of the phase (PH1) of the electrical signal comprises:
an angular phase shift (40) of the electrical signal (SG1b) in at least one cycle of the electrical signal.

8. Method according to claim 7, wherein the angular shift (40) is configured to cause a phase (PH1) inversion of the electrical signal (SG1b).

9. Method according to one of claims 1 to 4, wherein modulation (ML1) of the phase (PH1) of the electrical signal comprises a variation (60) in the period of the cycles of the electrical signal (SG1d) over time.

10. Method according to any one of the preceding claims, wherein modulation (ML1) of the phase is configured so that the electrical signal (SG1) supplies electrical energy at least equal to theoretical energy that would be supplied by the electrical signal if its phase were not disturbed by said phase modulation.

11. Method according to any one of the preceding claims, wherein the phase (PH1) is modulated (ML1) while maintaining the same frequency on each cycle of the electrical signal (SG1).

12. Method according to any one of the preceding claims, wherein the ultrasonic waves (W) are compression waves generating shear waves in a medium (M).

13. Method according to any one of the preceding claims, wherein said method is applied to medical ultrasound imaging.

14. Computer program (PG1) including instructions for carrying out the steps of a method according to any one of the preceding claims when said program is run by a computer (10).

15. Device (10) for processing an electrical signal (SG1) for an ultrasonic transmitting device (20), said processing device comprising:
a generation module (MD2) configured to generate an electrical signal (SG1) defining ultrasonic waves (W), the periodicity of the electrical signal being modified by modulation (ML1) of the phase (PH1) of the signal; and
a supply module (MD4) configured to supply the electrical signal to the ultrasonic transmitting device (20) to cause the ultrasonic waves (W) to be emitted.
